(19) United States
(12) Patent Application Publication
(10) Pub. No.: US 2009/0240114 A1
(43) Pub. Date: Sep. 24, 2009

(54) BREAST CANCER DIAGNOSIS APPARATUS

(75) Inventors: Jong-Moon LEE, Chungbuk (KR); Hyuk-Je Kim, Daejon (KR); Younju Lee, Gyeonggi-do (KR); Soon-Ik Jeon, Daejon (KR)

Correspondence Address: RABIN & Berdo, PC, 1101 14TH STREET, NW, SUITE 500, WASHINGTON, DC, 20005, US

(73) Assignee: ELECTRONICS AND TELECOMMUNICATIONS RESEARCH INSTITUTE, Daejon (KR)

(21) Appl. No.: 12/186,606

(22) Filed: Aug. 6, 2008

(30) Foreign Application Priority Data
Mar. 18, 2008 (KR) 10-2008-0024825

(51) Int. Cl. A61B 5/00

(52) U.S. Cl. 600/300

(57) ABSTRACT

A breast cancer diagnosis apparatus controls supply and flow rate of fluid to rapidly and effectively supply the fluid. As a result, time for diagnosing the breast cancer is shortened. Also, by preventing overflow or shortage of the fluid, the breast cancer diagnosis apparatus accurately diagnoses the breast cancer. A breast cancer diagnosis apparatus includes a fluid supply unit, and a supply control unit for controlling the fluid supplied from the fluid supply unit.

### CROSS-REFERENCE TO RELATED APPLICATIONS

The present invention claims priority of Korean Patent Application No. 10-2008-0024825, filed on Mar. 18, 2008, which is incorporated herein by reference.

### BACKGROUND OF THE INVENTION

1. Field of the Invention

The present invention relates to an apparatus for diagnosing a breast cancer based on a heritability difference between a breast with normal tissue and that with cancer cells; and, more particularly, to a breast cancer diagnosis apparatus for accurately diagnosing a breast cancer in a short period of time by effectively controlling supply and speed of fluid.

This work was supported by the IT R&D program of MIC/IITA [2007-F-043-01, “Study on Diagnosis and Protection Technology based on EM”].

2. Description of Related Art

Recently, as medical and consciousness levels increase, early discovery and complete recovery rates of breast cancer have been increased. However, a development rate of the breast cancer has been relatively increased due to westernized way living, change in diet pattern, and environmental pollution. Also, westernized life styles represented by a single life, late marriage, an increase in first delivery age, a decrease in fertility rate, and decrease in breast milk feeding rate increase the development of the breast cancer.

The recovery rate of the breast cancer is greatly different according to stages of the cancer. To be specific, the recovery rate of the breast cancer in the third and fourth stages is much lower than that in the first and second stages. More than half patients discover their breast cancer when the cancer is in the third or fourth stage. Thus, it is urgently needed to develop a breast cancer diagnosis method. Typical breast cancer diagnosis methods use an x-ray, supersonic wave, and Magnetic Resonance Imaging (MRI).

The x-ray is most widely used today among the above described methods for diagnosing the breast cancer. To take an x-ray picture, the breast is brought to a tight contact to a plastic board. The test procedure is relatively simple. However, image of the breast may be blurred unless the patient pushes her breast against the plastic board because the breast tissues are soft. That is, pushing the breast with a pressure is very important to get a clear image. Thus, the patient may feel shame during the test.

Also, x-ray picture cannot distinctively show a lump or a tumor that can be detected by a supersonic wave test when the breasts are dense breasts or heterogeneously dense breasts that look white due to high-density fat tissues, connective tissues, and mammary gland tissues. Furthermore, a minimum detection image size is more than approximately 5 mm. Thus, it is difficult to detect micro-calcification in an early stage of the cancer and diagnosis accuracy rate is not more than about 80%.

According to the method using supersonic waves, sound waves reflecting from objects are gathered to acquire an image of a breast and the breast image is used to detect a cyst or a solid tumor and determine size, shape, and density of the tumor. As a result, it is possible to decide whether a tumor is a cancer or a benign tumor. The method using supersonic waves can be used for a case when x-ray photographing is impossible or when it is difficult to make a diagnosis. For instance, the method using supersonic waves is used for women having an inflammatory breast disease, a dense breast tissues or pregnant women. However, the method using supersonic waves, too, hardly detect the micro-calcification. Thus, the method using supersonic waves is simultaneously used in combination with the x-ray image photographing.

MRI is efficient to figure out spread and stage of a breast cancer. However, it takes expensive cost, long image-picturing time, and complicated procedure. Furthermore, the MRI test is carried out in a limited space so that it is not suitable for the claustrophobic.

To solve the problems, recently, a technology using a microwave imaging is used. When normal cells are changed into cancer cells, the heritability of the cancer cells rapidly increases compared to that of the normal cells. Thus, it is possible to diagnose the breast cancer by sensing and imaging the heritability of the normal cell and the cancer cell.

FIG. 1 is a schematic diagram of a breast cancer diagnosis apparatus 100 and a separate fluid supply apparatus 200. The breast cancer diagnosis apparatus 100 includes a measuring unit 10 filled with fluid having a heritability of a normal breast. The fluid is supplied to the measuring unit 10 through a fluid supply tube 12 from the fluid supply apparatus 200. A sensing unit 11 disposed in the measuring unit 10 generates electromagnetic waves to sense the heritability in a breast. The sensed data is transmitted to a computer (not shown) and then imaged.

However, the typical breast cancer diagnosis apparatus uses a pump to supply the fluid even though pressure and airtight of the fluid are important. Since it is hard to control supply speed of the fluid, the fluid flows slowly and ineffectively. The measuring unit 10 cannot sense an overflow or a shortage of the fluid. That is, the typical breast cancer diagnosis apparatus cannot accurately diagnose the breast cancer.

### SUMMARY OF THE INVENTION

An embodiment of the present invention is directed to providing a breast cancer diagnosis apparatus that can shorten time for diagnosing the breast cancer and sense and control the supply and flow rate of the fluid to rapidly and effectively supply the fluid by preventing the overflow or the shortage of the fluid, the breast cancer diagnosis apparatus accurately diagnoses the breast cancer.

In accordance with an aspect of the present invention, there is provided a breast cancer diagnosis apparatus including a fluid supply unit, and a supply control unit for controlling the fluid supplied from the fluid supply unit.

The breast cancer diagnosis apparatus includes a fluid quantity detecting sensor for sensing quantity of fluid and a controller for controlling the fluid supply unit based on sensing information of the fluid quantity detecting sensor.

The breast cancer diagnosis apparatus includes a fluid supply unit for supplying the fluid to a measuring unit and a driving unit driving the fluid supply unit under control of the controller.

The breast cancer diagnosis apparatus also includes a cylinder storing the fluid and a piston moving to and fro in the cylinder to supply or withdraw to or from a measuring unit of the breast cancer diagnosis apparatus.

The driving unit of the present invention is a motor connected to the piston through a moving axis to move the piston to and fro.

Other objects and advantages of the present invention can be understood by the following description, and become apparent with reference to the embodiments of the present invention. Also, it is obvious to those skilled in the art to which the present invention pertains that the objects and advantages of the present invention can be realized by the means as claimed and combinations thereof.

### BRIEF DESCRIPTION OF THE DRAWINGS

FIG. 1 is a schematic diagram of a breast cancer diagnosis apparatus 100 and a separate fluid supply apparatus 200.

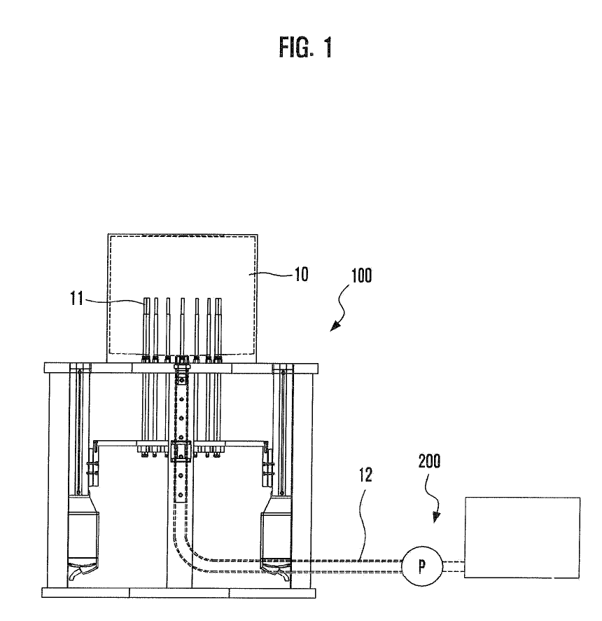

FIG. 2 is a schematic diagram of a breast cancer diagnosis apparatus and a driving unit in accordance with an embodiment of the present invention.

### DESCRIPTION OF SPECIFIC EMBODIMENTS

The advantages, features and aspects of the invention will become apparent from the following description of the embodiments with reference to the accompanying drawings, which is set forth hereinafter.

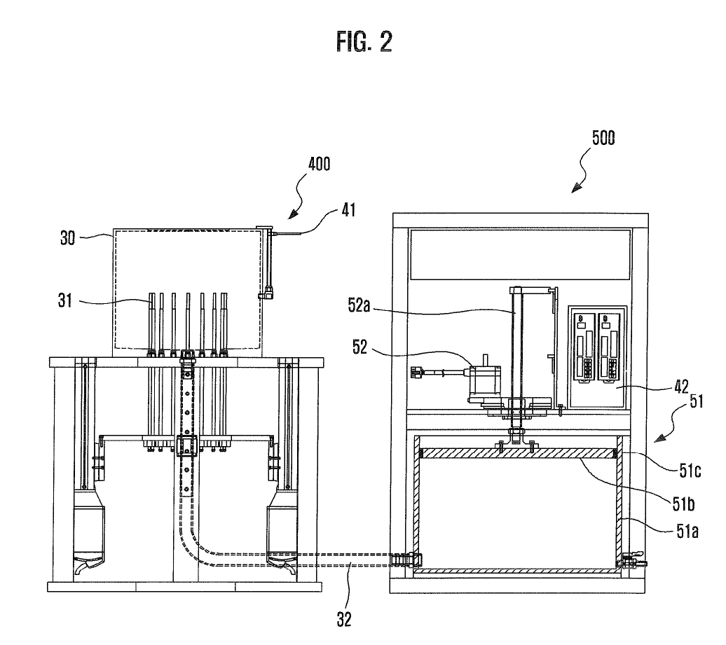

FIG. 2 is a schematic diagram of a breast cancer diagnosis apparatus and a driving unit in accordance with an embodiment of the present invention.

Referring to FIG. 2, the breast cancer diagnosis apparatus of the present invention includes a fluid supply apparatus 500. Since the fluid supply unit 500 supplying the fluid is controlled by a supply control unit 400. Thus, it is possible to rapidly and effectively transmit the fluid and prevent an overflow or a shortage of the fluid to accurately diagnose the breast cancer.

The supply control unit 400 includes a fluid quantity detecting sensor 41 disposed at the end of a measuring unit 30 to sense the fluid quantity supplied to the measuring unit 30.

A controller 42 controls a driving unit of the fluid supply apparatus 500 based on the sensing information which is fluid quantity of the fluid quantity detecting sensor 41. Thus, the controller 42 can control the quantity and flow rate of the fluid supplied to the measuring unit 30.

The fluid supply apparatus 500 supplies fluid based on a control signal from a supply control apparatus 400. The fluid supply apparatus 500 includes a fluid supply unit 51 and a driving unit.

The fluid supply unit 51 supplies the fluid to the measuring unit 30. Various technologies such as a pump system or a spray system controlled by the controller 42 can be applied to the fluid supply unit 51. In this embodiment, cylinder 51A storing the fluid and piston 51B are applied to accurately control the supply pressure and the flow rate of the fluid. The piston 51B moves to and fro in the cylinder 51A and supplies or withdraws the fluid in the cylinder 51A to or from the measuring unit 30 through the fluid supply tube 32.

Furthermore, in this embodiment, an O-ring 51C is connected to a circumferential surface of the piston 51B to simultaneously secure an airtight and a pressure between the piston 51B and the cylinder 51A.

The driving unit drives the fluid supply unit 51. Hereinafter, the embodiment of the driving unit is described referring to FIG. 2.

The driving unit controlled by the controller 42 can apply various technologies. In this embodiment, a motor 52 is applied for a utility efficiency in consideration of a production cost of the apparatus.

When the motor 52 is applied, referring to FIG. 2, the motor 52 controlled by the controller 42 of the supply control apparatus 400 is connected to the piston 51B through a moving axis 52A and a gear unit (not shown). As a result, the piston 51B moves to and fro using a rotation force of the motor 52.

The breast cancer diagnosis apparatus controls the supply and flow rate of the fluid to rapidly and effectively supply the fluid. As a result, time for diagnosing the breast cancer is shortened. Also, by preventing the overflow or the shortage of the fluid, the breast cancer diagnosis apparatus accurately diagnoses the breast cancer.

While the present invention has been described with respect to the specific embodiments, it will be apparent to those skilled in the art that various changes and modifications may be made without departing from the spirit and scope of the invention as defined in the following claims.

What is claimed is:

1. A breast cancer diagnosis apparatus, comprising:
a fluid supply unit; and
a supply control unit for controlling the fluid supplied from the fluid supply unit.

2. The apparatus of claim 1, wherein the fluid supply unit comprises:
a fluid quantity detecting sensor for sensing quantity of fluid; and
a controller for controlling the fluid supply unit based on sensing information of the fluid quantity detecting sensor.

3. The apparatus of claim 1, wherein the fluid supply unit comprises:
a fluid supply unit for supplying the fluid to a measuring unit; and
a driving unit for driving the fluid supply unit under control of the controller.

4. The apparatus of claim 3, wherein the fluid supply unit comprises:
a cylinder storing the fluid; and
a piston moving to and fro in the cylinder to supply or withdraw to or from a measuring unit of the breast cancer diagnosis apparatus.

5. The apparatus of claim 3, wherein the driving unit is a motor connected to the piston through a moving axis to move the piston to and fro.

6. The apparatus of claim 4, wherein an O-ring is connected to a circumferential surface of the piston.

7. The apparatus of claim 4, wherein the fluid stored in the cylinder is supplied to the measuring unit through a fluid supply tube.
